SEQUENCE LISTING (31 sequences)

SEQ ID NO: 1 - DNA, 2500 bp, Homo sapiens
agtcacatggggcccctaaactccctgaagctccccctgtccccccggcctgagcatctgccctgtggttgtattccagccatgaaagcccaccccaaggagatggtgcctctcatgggcaagagagttgctgcccccagtgggaaccctgccgtcctgccagagaagaggccggcggagatcacccccacaaagaagagtgcacacttcttcctggagatagaagggtttgaacccaaccccacagttgccaagacctctcctcctgtcttctccaagcccatggattccaacatccggcagtgcatctctggtaactgtgatgacatggactccccccagtctcctcaagatgatgtgacagagaccccatccaatcccaacagccccagtgcacagctggccaaggaagagcagaggaggaaaaagaggcggctgaagaagcgcatctttgcagccgtgtctgagggctgcgtggaggagttggtagagttgctggtggagctgcaggagctttgcaggcggcgccatgatgaggatgtgcctgacttcctcatgcacaagctgacggcctccgacacggggaagacctgcctgatgaaggccttgttaaacatcaaccccaacaccaaggagatagtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgtgtttttgcttggatttggagtagccttggcctcgctgatcgagaagtgtcccaaagacaacaaggactgcagctcctacggcagcttcagcgacgcagtgctggaactcttcaagctcaccataggcctgggtgacctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacagatttcaacaaaatccaagattcttccaggaacaacagcaaaaccactctcaatgcatttgaagaagtcgaggaattcccggaaacctcggtgtagaagcggaacccagagctggtgtgcgcgtgcgctgtctggcgctgca

SEQ ID NO: 2 - protein, 790 aa, Homo sapiens
MKAHPKEMVPLMGKRVAAPSGNPAVLPEKRPAEITPTKKSAHFFLEIEGFEPNPTVAKTSPPVFSKPMDSNIRQCISGNCDDMDSPQSPQDDVTETPSNPNSPSAQLAKEEQRRKKRRLKKRIFAAVSEGCVEELVELLVELQELCRRRHDEDVPDFLMHKLTASDTGKTCLMKALLNINPNTKEIVRILLAFAEENDILGRFINAEYTEEAYEGQTALNIAIERRQGDIAALLIAAGADVNAHAKGAFFNPKYQHEGFYFGETPLALAACTNQPEIVQLLMEHEQTDITSRDSRGNNILHALVTVAEDFKTQNDFVKRMYDMILLRSGNWELETTRNNDGLTPLQLAAKMGKAEILKYILSREIKEKRLRSLSRKFTDWAYGPVSSSLYDLTNVDTTTDNSVLEITVYNTNIDNRHEMLTLEPLHTLLHMKWKKFAKHMFFLSFCFYFFYNITLTLVSYYRPREEEAIPHPLALTHKMGWLQLLGRMFVLIWAMCISVKEGIAIFLLRPSDLQSILSDAWFHFVFFIQAVLVILSVFLYLFAYKEYLACLVLAMALGWANMLYYTRGFQSMGMYSVMIQKVILHDVLKFLFVYIVFLLGFGVALASLIEKCPKDNKDCSSYGSFSDAVLELFKLTIGLGDLNIQQNSKYPILFLFLLITYVILTFVLLLNMLIALMGETVENVSKESERIWRLQRARTILEFEKMLPEWLRSRFRMGELCKVAEDDFRLCLRINEVKWTEWKTHVSFLNEDPGPVRRTDFNKIQDSSRNNSKTTLNAFEEVEEFPETSV

SEQ ID NO: 3 - DNA, 25 bp, Artificial Sequence
  Oligonucleotide primer
gaagacgcacgtctccttccttaac

SEQ ID NO: 4 - DNA, 25 bp, Artificial Sequence
  Oligonucleotide primer
ttcataggcctcctctgtgtactcg

SEQ ID NO: 5 - DNA, 23 bp, Artificial Sequence
  Oligonucleotide primer
gcggtagtacgagacgagggtca

SEQ ID NO: 6 - DNA, 23 bp, Artificial Sequence
  Oligonucleotide primer
catgagatgctgaccctggagcc

SEQ ID NO: 7 - DNA, 25 bp, Artificial Sequence
  Oligonucleotide primer
agtacctgttcgtcttacaggcccc

SEQ ID NO: 8 - DNA, 24 bp, Artificial Sequence
  Oligonucleotide primer
gcgtggaggagttggtagagttgc

SEQ ID NO: 9 - DNA, 21 bp, Artificial Sequence
  Oligonucleotide primer
aggcacatcctcatcatggcg

SEQ ID NO: 10 - DNA, 24 bp, Artificial Sequence
  Oligonucleotide primer
atgaaagcccaccccaaggagatg

SEQ ID NO: 11 - DNA, 24 bp, Artificial Sequence
  Oligonucleotide primer
accagagatgcactgccggatgtt

SEQ ID NO: 12 - DNA, 24 bp, Artificial Sequence
  Oligonucleotide primer
cgcttcttcagccgcctctttttc

SEQ ID NO: 13 - DNA, 24 bp, Artificial Sequence
  Oligonucleotide primer
tccatgggcttggagaagacagga

SEQ ID NO: 14 - DNA, 24 bp, Artificial Sequence
  Oligonucleotide primer
agatgcactgccggatgttggaat

SEQ ID NO: 15 - DNA, 27 bp, Artificial Sequence
  Oligonucleotide primer
ccatccatctctcccagccaagccgac

SEQ ID NO: 16 - DNA, 27 bp, Artificial Sequence
  Oligonucleotide primer
cctcctcccccaggatatccagcttac

SEQ ID NO: 17 - DNA, 27 bp, Artificial Sequence
  Oligonucleotide primer
aattggtaaaaccagaggcttcacccg

SEQ ID NO: 18 - DNA, 28 bp, Artificial Sequence
  Oligonucleotide primer
ttgctctgtggaggtcaaaactcttgga

SEQ ID NO: 19 - DNA, 28 bp, Artificial Sequence
  Oligonucleotide primer
tgctgggctctcctaggaccatagcatt

SEQ ID NO: 20 - DNA, 28 bp, Artificial Sequence
  Oligonucleotide primer
gaagaaggattggtgaactgggaaggga

SEQ ID NO: 21 - DNA, 23 bp, Artificial Sequence
  Oligonucleotide primer
gttgatgaacctgcccaggatgt

SEQ ID NO: 22 - DNA, 24 bp, Artificial Sequence
  Oligonucleotide primer
tcaaccccaacaccaaggagatag

SEQ ID NO: 23 - protein, 25 aa, homo sapiens
MGPLNSLKLPLSPRPEHLPCGCIPA

SEQ ID NO: 24 - DNA, 20 bp, Artificial Sequence
  Oligonucleotide primer
gggccttcttcaaccccaag

SEQ ID NO: 25 - DNA, 20 bp, Artificial Sequence
  Oligonucleotide primer
aacttcctggacaggctccg

SEQ ID NO: 26 - DNA, 45 bp, Artificial Sequence
  Oligonucleotide probe
gcaaaggcaagcaggatccgcactatctccttggtgttggggtgg

SEQ ID NO: 27 - DNA, 45 bp, Artificial Sequence
  Oligonucleotide probe
atgtccgtctgctcgtgctccatcagcagctgcacaatctcgggc

SEQ ID NO: 28 - DNA, 45 bp, Artificial Sequence
  Oligonucleotide probe
gcttgaagagttccagcactgcgtcgctgaagctgccgtaggagc

SEQ ID NO: 29 - DNA, 2476 bp, Mus sp.
cagcatatgccttaggctccagcaatgaatgcccactccaaggagatggcgcccctcatgggcaaaagaaccacggcacctggcgggaaccctgttgtactgacagagaagaggccagcagatctcacccccaccaagaagagtgcacacttcttcctggagatagaaggatttgagcccaaccccacggtcaccaagacctctccacccatcttctccaagccgatggactccaacatccggcagtgcctctctggcaactgtgatgacatggactctccccagtctcctcaggatgatgtgacagagaccccatccaatcccaacagtccgagcgcaaacctggccaaggaagaacagaggcagaagaagaagcgactgaagaagcgcatcttcgcggctgtgtccgagggctgcgtggaggagctgcgggaactcctacaggatctgcaggacctctgcaggaggcgccgcggcctggatgtgcctgacttcctcatgcacaagctgacagcctcagacaccgggaagacctgcctgatgaaggctttgctcaacatcaatcccaacaccaaagagatcgtgcggattctgcttgccttcgctgaggagaacgacatcctggacaggttcatcaacgctgagtacacggaagaggcctatgaagggcagacagcgctgaacatcgccatcgagcggcgccagggagacatcacagcagtgcttatagcagcgggtgctgacgtcaatgctcacgccaagggggtcttcttcaaccccaaataccagcatgaaggcttctattttggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgatgcagctggccgccaagatgggcaaggcggagatcctgaagtacatcctcagccgcgagatcaaggagaagcctctccggagcttgtccaggaagttcacggactgggcgtatgggcctgtgtcatcctcactctatgacctcaccaatgtagacacaacgacggataactctgtgctggaaatcatcgtctacaacaccaacattgataaccgacatgagatgctgaccctggagcctctgcatacgctgctacacacgaaatggaagaaatttgccaagtacatgttcttcttgtccttctgcttctatttcttctacaacatcaccctgacccttgtctcttactaccgtcctcgggaagatgaggatctcccacaccccttggccctgacacacaaaatgagttggcttcagctcctagggaggatgtttgtcctcatctgggccacatgcatctctgtgaaagaaggcattgccattttcctgctgagaccctccgatcttcagtccatcctgtcagatgcctggtttcactttgtcttttttgtccaagctgtacttgtgatactgtctgtattcttgtacttgtttgcctacaaagaatacctcgcctgcctcgtgctggccatggccctgggctgggcgaacatgctctactacacgagaggcttccagtctatgggcatgtacagcgtcatgatccagaaggtcattttgcatgatgtcctcaagttcttgtttgtttacatcctgttcttacttggatttggagtagcgctggcctcactgattgagaagtgctccaaggacaaaaaggactgcagttcctatggcagcttcagcgacgcggtgctggagctcttcaagctcaccataggcctgggcgacctgaacatccagcagaactccacctaccccatcctctttctcttcctactcatcacctatgtcatcctcaccttcgtcctcctcctcaacatgctcattgccctgatgggggagacggtggagaacgtctccaaagaaagtgagcggatctggcgcttgcagagagccaggaccatcttggagtttgagaaaatgttaccagaatggctgagaagcagattccgcatgggcgagctgtgcaaagtagcagatgaggacttccggctgtgtctgcggatcaacgaggtgaagtggacggaatggaaaacacacgtgtccttccttaatgaagacccgggacccataagacggacagatttaaacaagattcaagattcttccaggagcaatagcaaaaccaccctctatgcgtttgatgaattagatgaattcccagaaacgtcggtgtagatgcctggcttaaggcggatgtgaaccctgtggtttgatgctgcaggctgagccctggtgtgctgaagtgctatgcaga

SEQ ID NO: 30 - protein, 790 aa, Mus sp.
MNAHSKEMAPLMGKRTTAPGGNPVVLTEKRPADLTPTKKSAHFFLEIEGFEPNPTVTKTSPPIFSKPMDSNIRQCLSGNCDDMDSPQSPQDDVTETPSNPNSPSANLAKEEQRQKKKRLKKRIFAAVSEGCVEELRELLQDLQDLCRRRRGLDVPDFLMHKLTASDTGKTCLMKALLNINPNTKEIVRILLAFAEENDILDRFINAEYTEEAYEGQTALNIAIERRQGDITAVLIAAGADVNAHAKGVFFNPKYQHEGFYFGETPLALAACTNQPEIVQLLMEHEQTDITSRDSRGNNILHALVTVAEDFKTQNDFVKRMYDMILLRSGNWELETTRNNDGLTPMQLAAKMGKAEILKYILSREIKEKPLRSLSRKFTDWAYGPVSSSLYDLTNVDTTTDNSVLEIIVYNTNIDNRHEMLTLEPLHTLLHTKWKKFAKYMFFLSFCFYFFYNITLTLVSYYRPREDEDLPHPLALTHKMSWLQLLGRMFVLIWATCISVKEGIAIFLLRPSDLQSILSDAWFHFVFFVQAVLVILSVFLYLFAYKEYLACLVLAMALGWANMLYYTRGFQSMGMYSVMIQKVILHDVLKFLFVYILFLLGFGVALASLIEKCSKDKKDCSSYGSFSDAVLELFKLTIGLGDLNIQQNSTYPILFLFLLITYVILTFVLLLNMLIALMGETVENVSKESERIWRLQRARTILEFEKMLPEWLRSRFRMGELCKVADEDFRLCLRINEVKWTEWKTHVSFLNEDPGPIRRTDLNKIQDSSRSNSKTTLYAFDELDEFPETSV

SEQ ID NO: 31 - protein, 18 aa, Homo sapiens
RTDFNKIQDSSRNNSKTC

(19) United States
(12) Patent Application Publication
(10) Pub. No.: US 2004/0005581 A1
(43) Pub. Date: Jan. 8, 2004

(54) Human vanilloid receptor protein and polynucleotide sequence encoding same

(76) Inventors: Timothy Peter Bonnert, Much Hadham (GB); Luiz Miguel Quinn de Camargo, London (GB)

Correspondence Address: MERCK AND CO INC, P O BOX 2000, RAHWAY, NJ, 070650907

(21) Appl. No.: 10/301,477

(22) Filed: Nov. 21, 2002

(30) Foreign Application Priority Data
Nov. 23, 2001 (GB) 0128161.7

(51) Int. Cl. C12Q 1/68; C07H 21/04; C07K 14/705; A61K 48/00

(52) U.S. Cl. 435/6; 514/44; 530/350; 536/23.5

(57) ABSTRACT

The present invention discloses a human vanilloid receptor-like receptor (designated VR4) and a polynucleotide sequence encoding same.

The present invention is in the field of molecular biology; more particularly, the present invention relates to an amino acid sequence for a novel human vanilloid receptor-like receptor (hereinafter designated VR4) and a polynucleotide sequence encoding same.

According to the invention there is provided an isolated and purified polypeptide comprising the amino acid sequence of SEQ ID NO 2. In a second aspect of the invention there is provided an isolated and purified polynucleotide which encodes a polypeptide comprising the amino acid sequence of SEQ ID NO 2. In one embodiment, said polynucleotide comprises the nucleotide sequence SEQ ID NO 1. The invention further relates to a stable cell line expressing a recombinant VR4 receptor and the use of the cell line in a screening technique for the design and development of receptor-specific medicaments.

The present invention provides a unique nucleotide sequence which encodes a novel human vanilloid receptor-like receptor (VR4). The cDNA, hereinafter designated vr4, was identified and cloned as described in the Examples below.

The invention relates to the use of nucleotide and amino acid sequences of VR4 or its variants, in the diagnosis or treatment of activated, inflamed or diseased cells and/or tissues associated with its expression. Further aspects of the invention include the antisense DNA of vr4; cloning or expression vectors containing vr4; host cells or organisms genetically engineered so as to express VR4; host cells or organisms genetically engineered so as to remove, prevent or reduce expression of VR4; a method for the production and recovery of purified VR4 from host cells genetically engineered so as to express VR4; subcellular fractions of said host cells containing VR4; the purified VR4 protein itself; assays to identify modulators of signal transduction involving VR4; and antibodies to VR4.

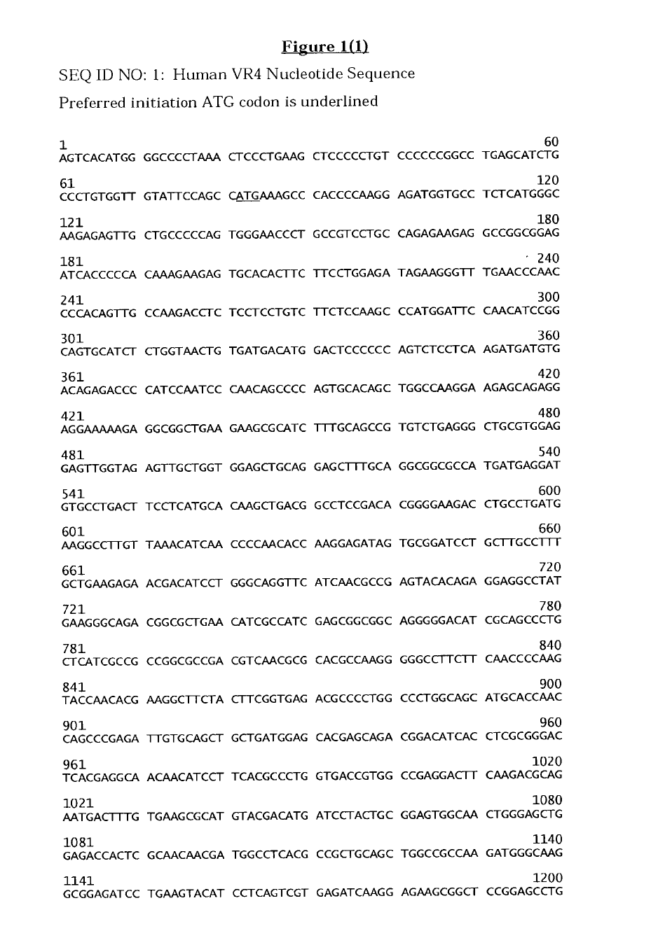

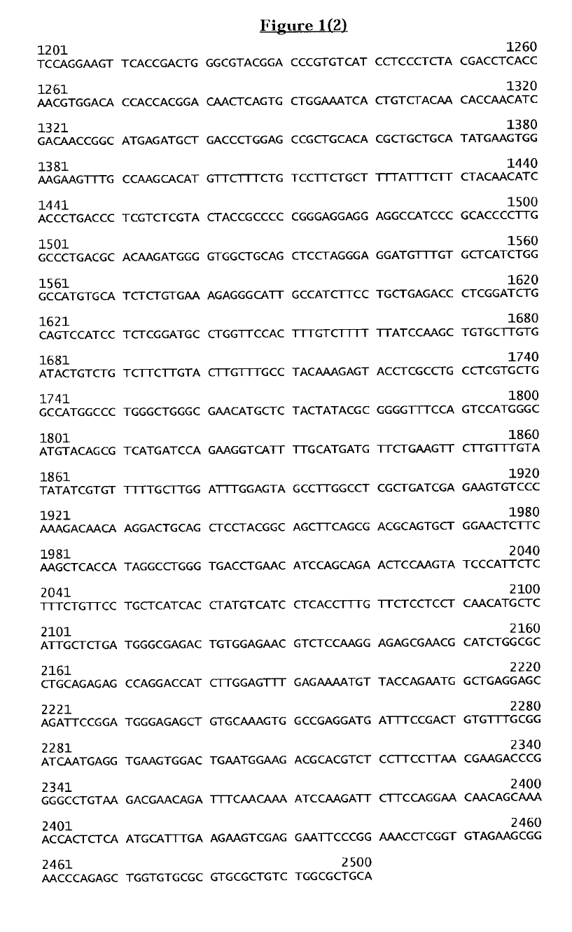

FIG. 1 shows the nucleotide sequence of the gene encoding human VR4 (SEQ ID NO: 1).

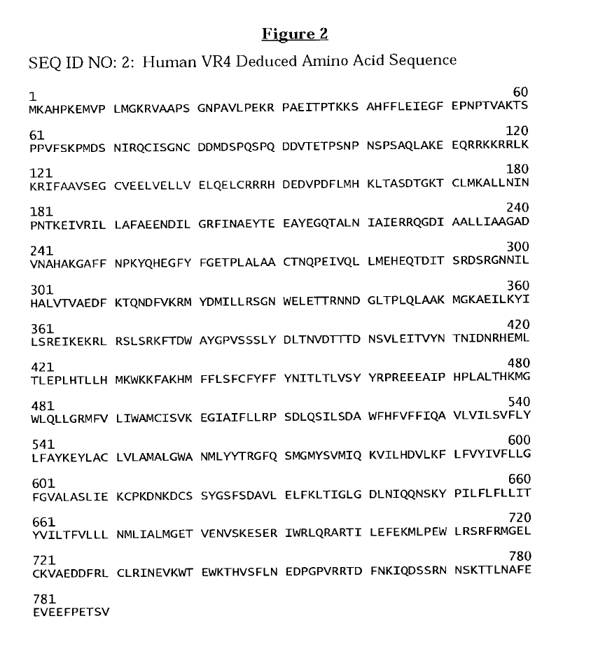

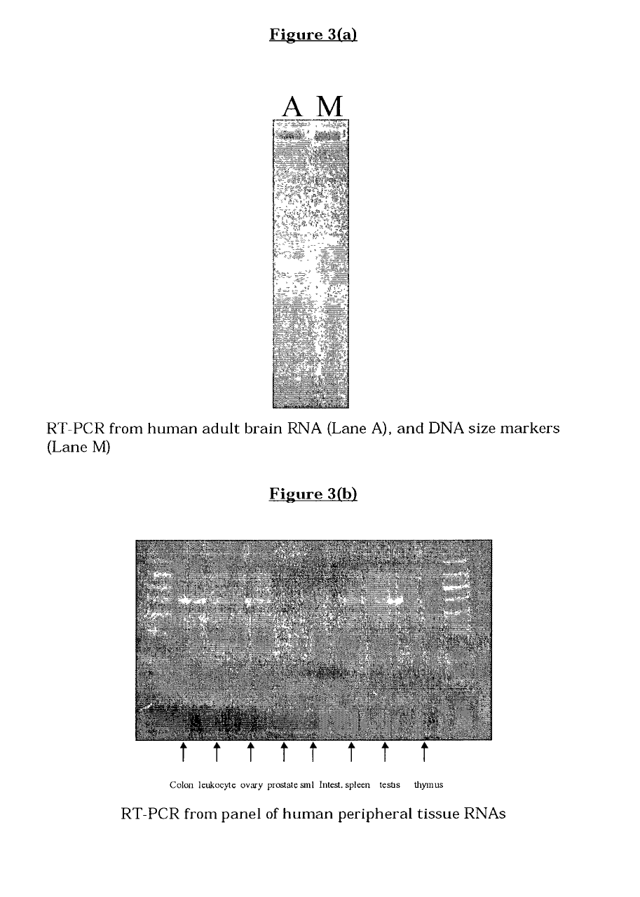

FIG. 2 shows the deduced amino acid sequence for human VR4 (SEQ ID NO: 2).

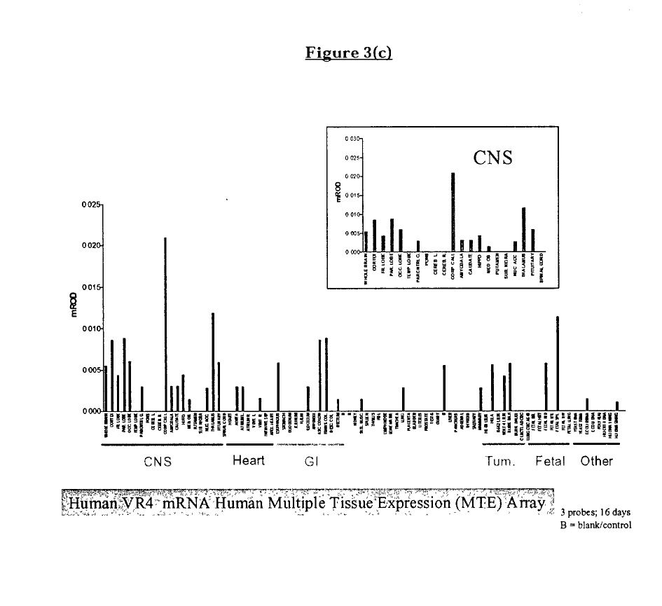

FIG. 3(a) shows the results of RT-PCR of a fragment of vr4 from RNA isolated from human adult brain.

FIGS. 3(b) and 3(c) show the distribution of mRNA encoding human VR4 as evidenced by, respectively, RT-PCR in a human adult tissues array (Clontech), and a Multiple Tissue DotBlot (Clontech).

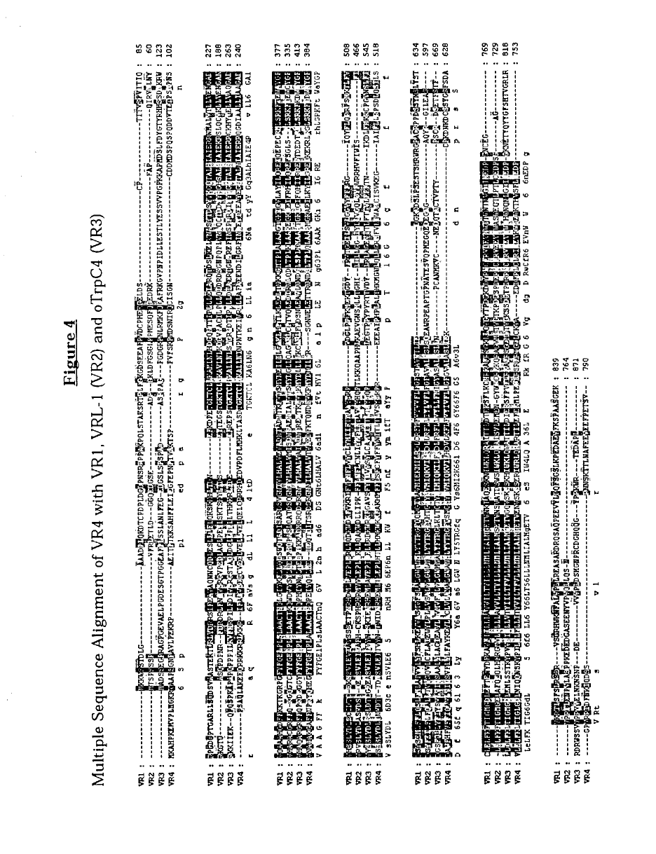

FIG. 4 shows a multiple sequence alignment of the genes encoding human VR1, VRL-1 (VR2), oTrpC4 (VR3) and VR4 receptors.

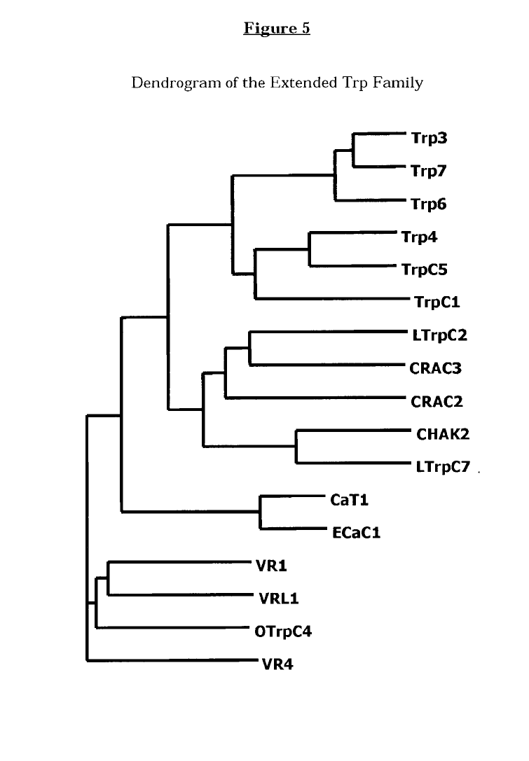

FIG. 5 shows a dendrogram illustrating sequence similarities in the extended Trp family.

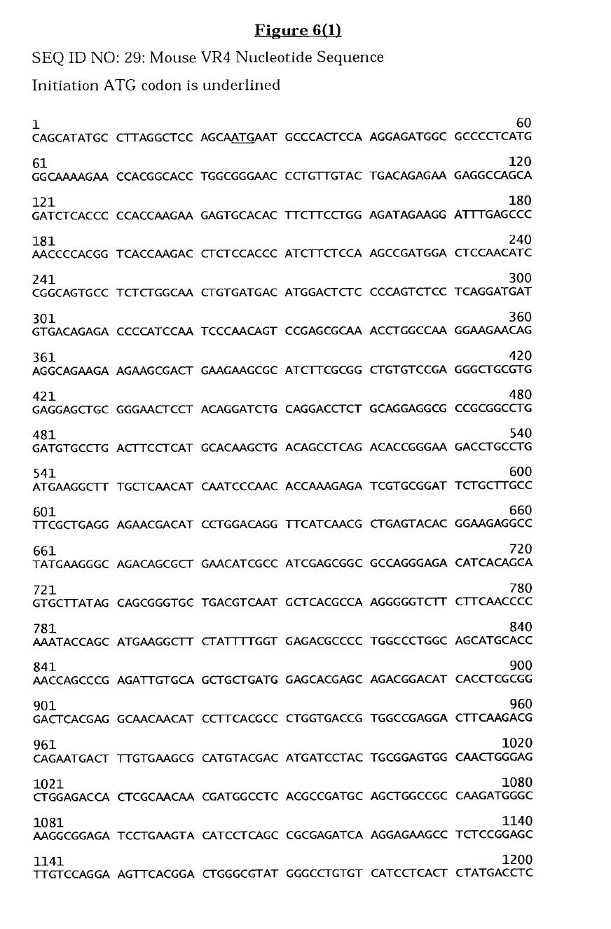

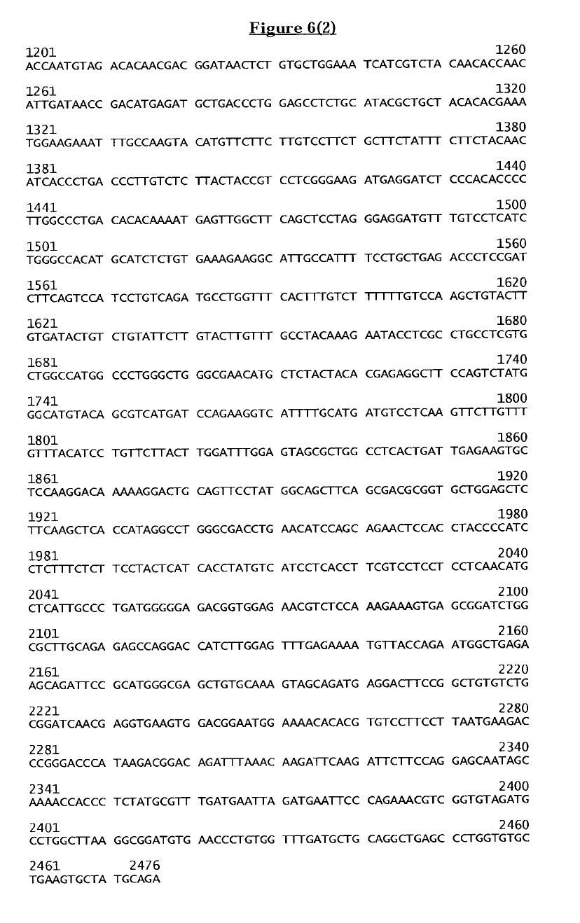

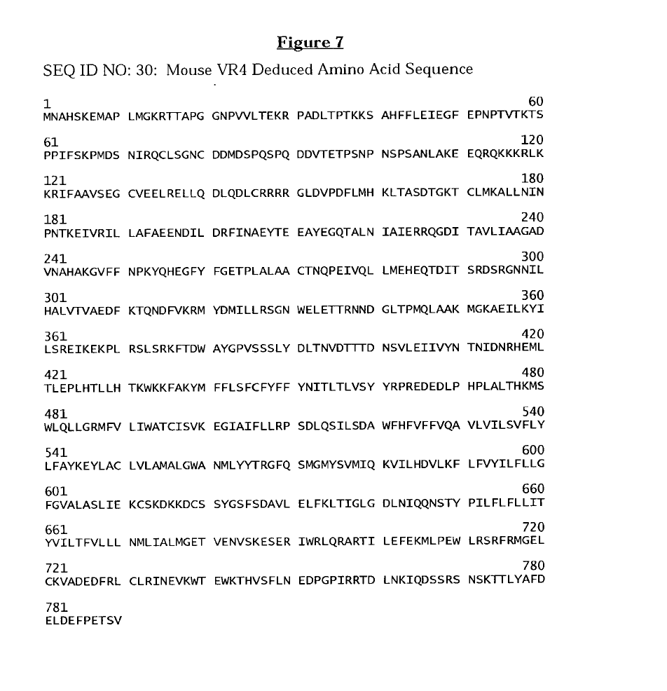

FIG. 6 shows the DNA sequence (SEQ ID NO: 29) encoding mouse VR4.

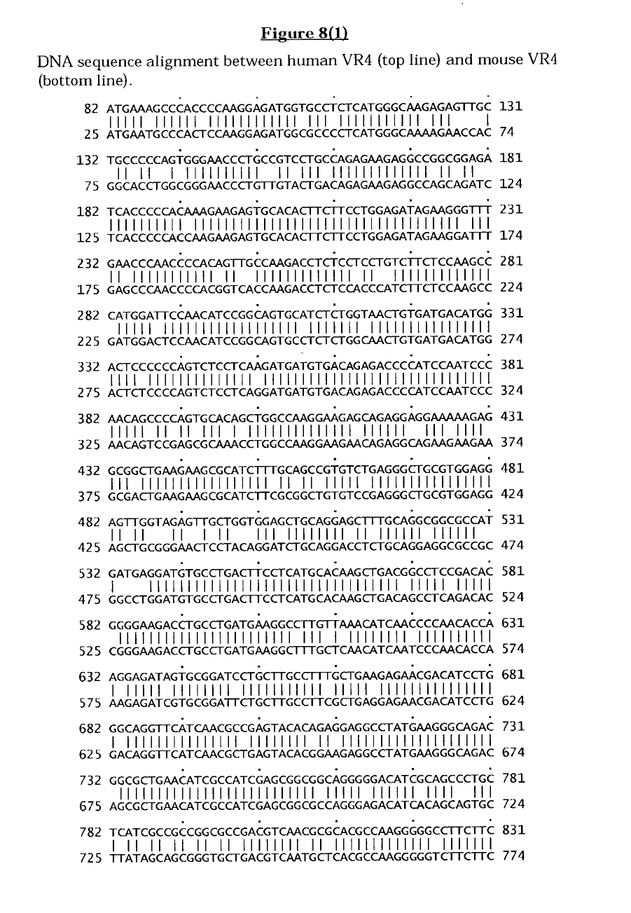

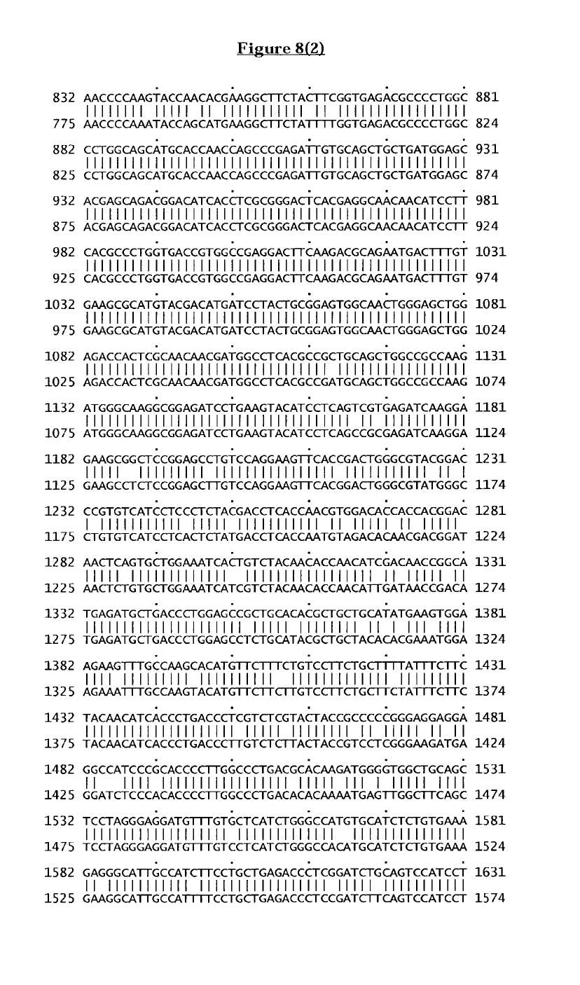

FIG. 7 shows the deduced amino acid sequence (SEQ ID NO: 30) of mouse VR4.

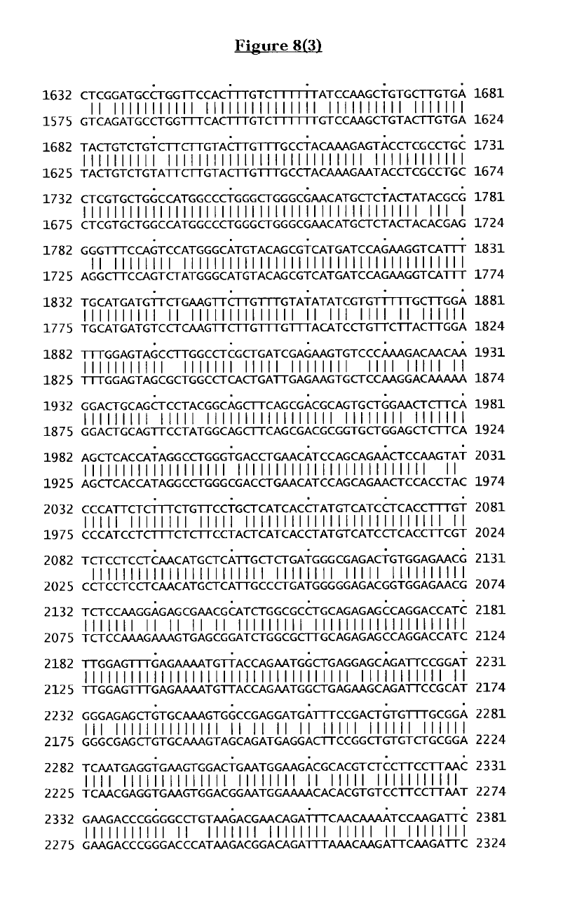

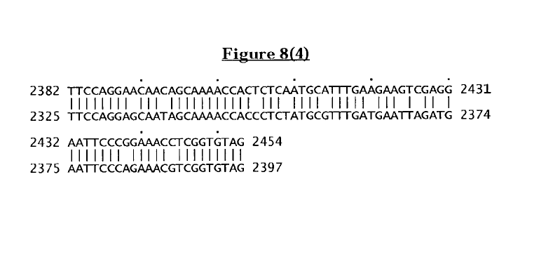

FIG. 8 shows a sequence alignment of the genes encoding human (top) and mouse (bottom) VR4.

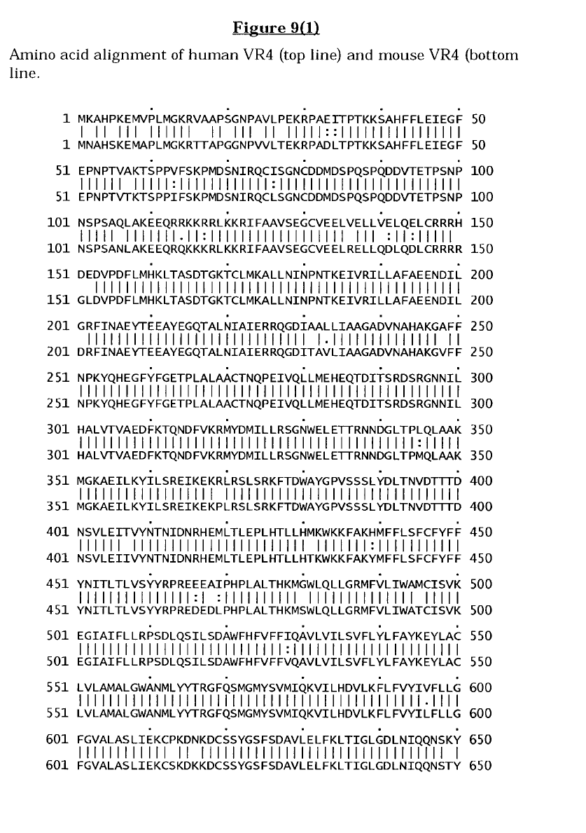

FIG. 9 shows a sequence alignment of the amino acid sequence of human (top) and mouse (bottom) VR4.

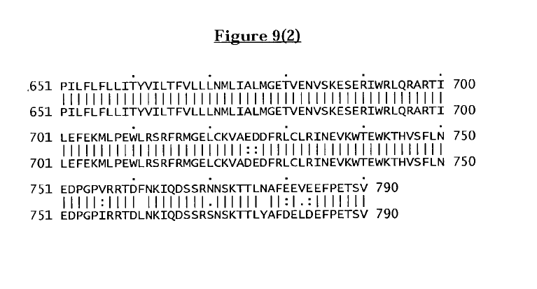

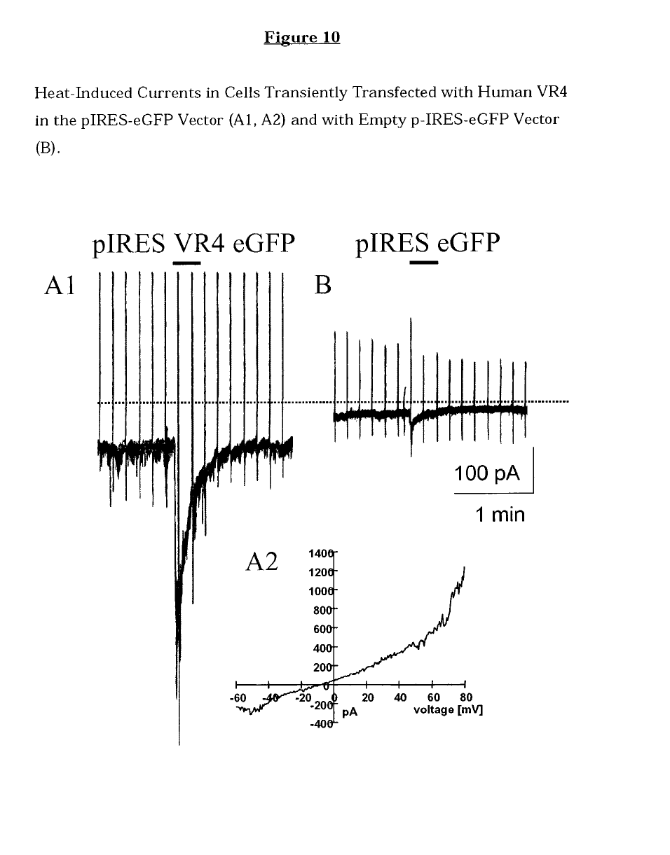

FIG. 10 shows the results of electrophysiological studies on CHO cells transiently transfected with human VR4 in the pIRES-eGFP vector (A1, A2) and CHO cells transiently transfected with empty pIRES-eGFP vector (B).

As used herein and designated by the upper case abbreviation, VR4, refers to a vanilloid receptor-like receptor protein in naturally occurring, recombinant or synthetic form and active fragments thereof which have the amino acid sequence of SEQ ID NO: 2. In vivo, the VR4 polypeptide may form part of a heteromeric complex with homologous receptor proteins. In one embodiment, the polypeptide VR4 is encoded by mRNAs transcribed from the cDNA, as designated by the lower case abbreviation, vr4, of SEQ ID NO: 1.

The novel human vanilloid receptor-like receptor VR4, which is the subject of this patent application, was discovered among the partial cDNA sequences present in the human High Throughput Genomic (HTG) phase 0-2 sequences of Genbank, contained in the Merck.HTG.Human database.

An “oligonucleotide” is a stretch of nucleotide residues which has a sufficient number of bases to be used as an oligomer, amplimer or probe in a polymerase chain reaction (PCR). Oligonucleotides are usually prepared by chemical synthesis. Their sequence is based on cDNA or genomic sequence information and are used to amplify, reveal or confirm the presence of a similar DNA or RNA in a particular cell or tissue. Oligonucleotides or oligomers comprise portions of a DNA sequence having at least about 10 nucleotides and as many as about 80 nucleotides, typically about 25 nucleotides.

“Probes” may be derived from naturally occurring or recombinant single- or double-stranded nucleic acids or be chemically synthesised. They are useful in detecting the presence of identical or similar sequences.

A “portion” or “fragment” of a polynucleotide or nucleic acid comprises all or part of the nucleotide sequence having fewer nucleotides than about 6 kb, preferably fewer than about 1 kb which can be used as a probe. Such probes may be labelled with reporter molecules using nick translation, Klenow fill-in reaction, PCR or other methods well known in the art. After pre-testing to optimise reaction conditions and to eliminate false positives, nucleic acid probes may be used in Southern, Northern or in situ hybridizations to determine whether DNA or RNA encoding VR4 is present in a cell type, tissue, or organ.

“Reporter” molecules are those radionuclides, enzymes, fluorescent, chemiluminescent, or chromogenic agents which associate with, establish the presence of, and may allow quantification of a particular nucleotide or amino acid sequence.

“Recombinant nucleotide variants” encoding VR4 may be synthesised by making use of the “redundancy” in the genetic code. Various codon substitutions, such as the silent changes which produce specific restriction sites or codon usage-specific mutations, may be introduced to optimise cloning into a plasmid or viral vector or expression in a particular prokaryotic or eukaryotic host system, respectively.

“Chimeric” molecules may be constructed by introducing all or part of the nucleotide sequence of this invention into a vector containing one or more additional nucleotide sequences which might be expected to change any one (or more than one) of the following VR4 characteristics: cellular location, distribution, ligand-binding affinities, interchain affinities, degradation/turnover rate, signalling, etc.

“Active” refers to those forms, fragments, or domains of any VR4 polypeptide which retain the biological and/or antigenic activities of any naturally occurring VR4.

“Naturally occurring VR4” or “native VR4” refers to the relevant polypeptide produced by cells which have not been genetically engineered and specifically contemplates various polypeptides arising from post-translational modifications of the polypeptide including but not limited to acetylation, carboxylation, glycosylation, phosphorylation, lipidation and acylation.

“Derivative” refers to those polypeptides which have been chemically modified by such techniques as ubiquitination, labelling (see above), pegylation (derivatization with polyethylene glycol), and chemical insertion or substitution of amino acids such as ornithine which do not normally occur in human proteins.

“Recombinant polypeptide variant” refers to any polypeptide which differs from naturally occurring VR4 by amino acid insertions, deletions and/or substitutions, created using recombinant DNA techniques. Guidance in determining which amino acid residues may be replaced, added or deleted without abolishing activities or interest may be found by comparing the sequence of VR4 with that of related polypeptides and minimizing the number of amino acid sequence changes made in highly conserved regions.

Amino acid “substitutions” are conservative in nature when they result from replacing one amino acid with another having similar structural and/or chemical properties, such as the replacement of a leucine with an isoleucine or valine, an aspartate with a glutamate, or a threonine with a serine.

“Insertions” or “deletions” are typically in the range of about 1 to 5 amino acids. The variation allowed may be experimentally determined by producing the peptide synthetically or by systematically making insertions, deletions, or substitutions of nucleotides in the vr4 sequence using recombinant DNA techniques.

An “oligopeptide” is a short stretch of amino acid residues and may be expressed from an oligonucleotide. It may be functionally equivalent to and the same length as (or considerably shorter than) a “fragment”, “portion”, or “segment” of a polypeptide. Such sequences comprise a stretch of amino acid residues of at least about 5 amino acids and often about 17 or more amino acids, typically at least about 9 to 13 amino acids, and of sufficient length to display biological and/or antigenic activity.

“Inhibitor” is any substance which retards or prevents a chemical or physiological reaction or response. Common inhibitors include but are not limited to antisense molecules, antibodies, channel blockers and antagonists.

“Standard” expression is a quantitative or qualitative measurement for comparison. It is based on a statistically appropriate number of normal samples and is created to use as a basis of comparison when performing diagnostic assays, running clinical trials, or following patient treatment profiles.

The present invention provides a nucleotide sequence uniquely identifying a novel human vanilloid receptor-like receptor. The nucleic acids (vr4), polypeptides (VR4) and antibodies to VR4 are useful in diagnostic assays which survey for increased or decreased receptor production or function. A diagnostic test for excessive expression of VR4 can accelerate diagnosis and proper treatment of abnormal conditions associated with pain, especially heat-mediated pain, arthritis pain and neuropathic pain, inflammation, neurodegeneration such as that associated with Alzheimer's disease, Parkinson's disease or ischemia, endocrine disorders, cardiovascular disease, bladder or bowel dysfunction, mood disorders (e.g. depression), obesity and cancer.

The nucleotide sequences encoding VR4 (or their equivalents arising from degeneracy in the genetic code) have numerous applications in techniques known to those skilled in the art of molecular biology. These techniques include use as hybridization probes, use in the construction of oligomers for PCR, use for chromosome and gene mapping, use in the recombinant production of VR4, and use in generation of antisense DNA or RNA, their chemical analogues and the like. Uses of polynucleotides encoding VR4 disclosed herein are exemplary of known techniques and are not intended to limit their use in any technique known to a person of ordinary skill in the art. Furthermore, the nucleotide sequences disclosed herein may be used in molecular biology techniques that have not yet been developed, provided the new techniques rely on properties of nucleotide sequences that are currently known, e.g. the triplet genetic code, specific base pair interactions, etc.

It will be appreciated by those skilled in the art that as a result of the degeneracy of the genetic code, a multitude of VR4-encoding nucleotide sequences may be produced. Some of these will only bear minimal homology to the naturally occurring nucleotide sequence governing the expression of naturally occurring VR4. The invention has specifically contemplated each and every possible variation of said naturally occurring nucleotide sequence which results in a polynucleotide encoding VR4 in accordance with the standard triplet genetic code. The variant given in FIG. 1 (SEQ ID NO: 1) is preferred.

Although the nucleotide sequences which encode VR4 (or its derivatives or variants) are preferably capable of hybridizing to the nucleotide sequence of the naturally occurring vr4 under stringent conditions, it may be advantageous to produce nucleotide sequences encoding VR4 or its derivatives possessing a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence encoding VR4 and/or its derivatives without altering the encoded amino acid sequence include the production of RNA transcripts having more desirable properties, such as a greater half-life, than transcripts produced from the naturally occurring sequence.

Nucleotide sequences encoding VR4 may be joined to a variety of other nucleotide sequences by means of well established recombinant DNA-techniques (Sambrook J et al (1989) Molecular Cloning: A Laboratory Manual, Cold Spring Harbor Laboratory, Cold Spring Habor N.Y.; or Ausubel F M et al (1989) Current Protocols in Molecular Biology, John Wiley & Sons, New York City). Useful nucleotide sequences for joining to vr4 include an assortment of cloning vectors such as plasmids, cosmids, lambda phage derivatives, phagemids, and the like. Vectors of interest include expression vectors, replication vectors, probe generation vectors, sequencing vectors, etc. In general, vectors of interest may contain an origin of replication functional in at least one organism, convenient restriction endonuclease sensitive sites, and selectable markers for one or more host cell systems.

Another aspect of the subject invention is to provide vr4-specific hybridization probes capable of hybridizing with naturally occurring nucleotide sequences encoding VR4. Such probes may also be used for the detection of similar sequences and should preferably contain at least 50% of the nucleotides from the vr4 sequence. The hybridization probes of the present invention may be derived from the nucleotide sequence presented as SEQ ID NO: 1 or from genomic sequences including promoters, enhancers or introns of the native gene. Hybridization probes may be labelled by a variety of reporter molecules using techniques well known in the art.

PCR as described U.S. Pat. Nos. 4,683,195; 4,800,195; and 4,965,188 provides additional uses for oligonucleotides based upon the nucleotide sequence which encodes VR4. Such probes used in PCR may be of recombinant origin, chemically synthesised, or a mixture of both. Oligomers may comprise discrete nucleotide sequences employed under optimised conditions for identification of vr4 in specific tissues or diagnostic use. The same two oligomers, a nested set of oligomers, or even a degenerate pool of oligomers may be employed under less stringent conditions for identification of closely related DNAs or RNAs.

Other means of producing specific hybridization probes for vr4 include the cloning of nucleotide sequences encoding VR4 or VR4 derivatives into vectors for the production of mRNA probes. Such vectors are known in the art, are commercially available and may be used to synthesise RNA probes in vitro by means of the addition of the appropriate RNA polymerase such as T7 or SP6 RNA polymerase and the appropriate reporter molecules.

It is possible to produce a DNA sequence, or portions thereof, entirely by synthetic chemistry. After synthesis, the nucleotide sequence can be inserted into any of the many available DNA vectors and their respective host cells using techniques which are well known in the art. Moreover, synthetic chemistry may be used to introduce mutations into the nucleotide sequence. Alternatively, a portion of sequence in which a mutation is desired can be synthesised and recombined with longer portion of an existing genomic or recombinant sequence.

The nucleotide sequence for vr4 can be used in an assay to detect or quantify disease states associated with abnormal levels of VR4 expression. The cDNA can be labelled by methods known in the art, added to a fluid, cell or tissue sample from a patient, and incubated under hybridising conditions. After the incubation period, the sample is washed with a compatible fluid which contains a reporter molecule. After the compatible fluid is rinsed off, the reporter molecule is quantitated and compared with a standard as previously defined. If VR4 expression is significantly different from standard expression, the assay indicates disease or other abnormality.

The nucleotide sequence for vr4 can be used to construct hybridisation probes for mapping the native gene or for identifying homologous gene sequences in other species. The gene may be mapped to a particular chromosome or to a specific region of a chromosome using well known mapping techniques. These techniques include in situ hybridisation of chromosomal spreads (Verma et al (1988) Human Chromosomes: A Manual of Basic Techniques, Pergamon Press, New York City), flow-sorted chromosomal preparations, or artificial chromosome constructions such as yeast artificial chromosomes (YACs), bacterial artificial chromosomes (BACs), bacterial P1 constructions or single chromosome cDNA libraries.

In situ hybridisation of chromosomal preparations and physical mapping techniques such as linkage analysis using established chromosomal markers are invaluable in extending genetic maps. Examples of genetic map data can be found in the yearly genome issue of Science (eg 1994, 265:1981f). Knowledge of the sequence and location of the corresponding gene in another species facilitates elucidation of its function, e.g. by the breeding of transgenic “knockout” animals or the use of antisense technology as described below.

The nucleotide sequence of the subject invention may also be used to detect differences in gene sequence between normal and carrier or affected individuals.

Knowledge of the correct, complete cDNA sequence of VR4 enables its use as a tool for antisense technology in the investigation of gene function. Oligonucleotides, cDNA or genomic fragments comprising the antisense strand of vr4 are used either in vitro or in vivo to inhibit expression of the mRNA. Such technology is now well known in the art, and antisense molecules can be designed at various locations along the nucleotide sequences. By treatment of cells or whole test animals with such antisense sequences, the gene of interest is effectively turned off. Frequently, the function of the gene is ascertained by observing behaviour at the intracellular, cellular, tissue or organismal level (eg. lethality, loss of differentiated function, changes in morphology, etc.)

In addition to using sequences constructed to interrupt transcription of a particular open reading frame, modifications of gene expression are obtained by designing antisense sequences to intron regions, promoter/enhancer elements, or even to transacting regulatory genes. Similarly, inhibition is achieved using Hogeboom base-pairing methodology, also known as “triple helix” base pairing.

Nucleotide sequences that are complementary to vr4 can be synthesised for antisense therapy. These antisense molecules may be DNA, stable derivatives of DNA such as LNA (locked nucleic acid), PNA (peptide nucleic acid), phosphorothioates or methylphosphonates, RNA, stable derivatives of RNA such as 2′-O-alkylRNA, or other VR4 receptor antisense mimetics. VR4 receptor antisense molecules may be introduced into cells by methods known in the art, including microinjection, liposome encapsulation and expression from vectors harbouring the antisense sequence. VR4 receptor antisense therapy may be particularly useful for the treatment of diseases where it is beneficial to reduce VR4 receptor activity.

VR4 receptor gene therapy may be used to introduce VR4 receptor into the cells of target organisms. The VR4 receptor gene can be ligated into, for example, viral vectors which mediate transfer of the VR4 receptor DNA by infection of recipient host cells. Suitable viral vectors include retrovirus, adenovirus, adeno-associated virus, herpes virus, vaccinia virus, polio virus and the like. Alternatively, VR4 receptor DNA can be transferred into cells for gene therapy by non-viral techniques such as receptor-mediated targeted DNA transfer using ligand-DNA conjugates or adenovirus-ligand-DNA conjugates, lipofection membrane fusion, and direct microinjection. These procedures and variants thereof are suitable for ex vivo as well as in vivo VR4 receptor gene therapy. VR4 receptor gene therapy may be particularly useful for the treatment of diseases where it is beneficial to elevate VR4 receptor expression.

Nucleotide sequences encoding VR4 may be used to produce a purified oligo- or polypeptide using well known methods of recombinant DNA technology. Goeddel (1990, Gene Expression Technology, Methods and Enzymology, Vol 185, Academic Press, San Diego Calif.) is one among many publications which teach expression of an isolated nucleotide sequence. Advantages of producing an oligo or polypeptide by recombinant DNA technology include obtaining adequate amounts for purification and the running of assays, and the availability of simplified purification procedures.

The cloned VR4 cDNA obtained as described above may be recombinantly expressed by molecular cloning into an expression vector containing a suitable promoter and other appropriate transcription regulatory elements and transferred into prokaryotic or eukaryotic host cells to produce recombinant VR4.

Expression vectors are defined herein as DNA sequences that are required for the transcription of cloned DNA and the translation of their mRNAs in an appropriate host. Such vectors can be used to express eukaryotic DNA in a variety of hosts such as bacteria, bluegreen algae, fungal cells, plant cells, insect cells and animal cells. Specifically-designed vectors allow the shuttling of DNA between hosts such as bacteria-yeast and bacteria-animal cells. An appropriately constructed expression vector contains an origin of replication for autonomous replication in host cells, selectable markers, a limited number of useful restriction enzyme sites, a potential for high copy number, and an active promoter. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids and viruses.

A variety of mammalian expression vectors may be used to express recombinant VR4 in mammalian cells. Commercially available mammalian expression vectors which may be suitable for recombinant VR4 expression include pMC1 (Stratagene), pcDNAI, pcDNAIamp, pcDNA3 (Invitrogen), pIRES, pIRES-eGFP (Clontech) and vaccinia virus transfer vector pTM1.

DNA encoding VR4 may also be cloned into an expression vector for expression in a host cell. Host cells may be prokaryotic or eukaryotic, including but not limited to bacteria, yeast, mammalian cells including but not limited to cell lines of human, bovine, porcine, monkey and rodent origin, and insect cells including but not limited to Sf9 and drosophila derived cell lines. Cell lines derived from mammalian species which may be suitable and which are commercially available include but are not limited to HEK293 (ATCC CRL 1573), tsa and Ltk cells, CV-1 (ATCC CCL 70), COS-1 (ATCC CRL 1650), COS-7 (ATCC CRL 1651), CHO K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), and MRC-5 (ATCC CCL 171).

The expression vector may introduced to host cells via any one of a number of techniques known to those skilled in the art, such as transformation, transfection, infection, protoplast fusion and electroporation. The cells containing expression vector are individually analyzed to determine whether they produce VR4 protein. Identification of VR4 expressing cells may be achieved by various means, such as immunological reactivity with anti-VR4 antibodies, or the presence of VR4-associated activity within the host cell.

Expression of VR4 DNA may also be achieved using synthetic mRNA produced in vitro. Synthetic mRNA can be efficiently translated in various cell-free systems such as wheat germ extracts and reticulocyte extracts, as well as in cell based systems, e.g. by microinjection into Xenopus oocytes.

The VR4 cDNA sequence(s) which yield(s) the optimum level(s) of receptor activity and/or protein production may be identified by constructing various VR4 cDNA molecules, such as the full length open reading frame of the VR4 cDNA and constructs containing portions of the cDNA which encode only selected domains, or rearranged domains, of the receptor protein. All such constructs can be designed to contain none, all, or portions of the 5′ and/or 3′ untranslated region of VR4 cDNA. VR4 activity and levels of protein expression can be determined following the introduction, singly or in combination, of these constructs into host cells. Following identification of the VR4 cDNA cassette yielding optimal expression in transient assays, this VR4 cDNA construct may be transferred to a variety of expression vectors (including recombinant viruses), including those for mammalian cells, plant cells, insect cells, oocytes, E. coli, fungal cells and yeast cells.

Transfected cells my be assayed for levels of VR4 receptor activity and/or levels of VR4 protein expression by known methods. Assessment of VR4 receptor activity typically involves the introduction of a labelled ligand (especially a radiolabelled ligand) to the cells and determination of the amount of specific binding of the ligand to the VR4-expressing cells. Binding assays for receptor activity are described in Frey et al., Eur. J. Pharmacol., 244, 239-250, 1993.

Levels of VR4 protein in host cells may be quantitated by a variety of techniques, such as proteomics, immunoaffinity and ligand affinity techniques. VR4-specific affinity beads or VR4-specific antibodies may be used to isolate ³⁵S-methionine labelled or unlabelled VR4 protein. Labelled protein may be analyzed by SDS-PAGE, while unlabelled protein may be analyzed by Western blotting, ELISA or RIA assays and protein arrays employing VR4 specific antibodies.

Following expression of VR4 in a host cell, VR4 protein may be recovered in active form, capable of binding VR4-specific ligands. Recombinant VR4 may be isolated and purified from cells or subcellular fractions by standard techniques of protein purification, such as detergent solubilisation, salt fractionation, ion exchange chromatography, hydroxylapatite adsorption chromatography and hydrophobic interaction chromatography.

In addition, recombinant VR4 can be separated from other cellular proteins by means of an immunoaffinity column made with monoclonal or polyclonal antibodies specific for full length nascent VR4 or polypeptide fragments of VR4.

Cells transformed with DNA encoding VR4 may be cultured under conditions suitable for expression of its extracellular, transmembrane or intracellular domains and recovery of such peptides from cell culture. VR4 (or any of its domains) produced by a recombinant cell may be secreted or may be contained intracellularly, depending on the particular genetic construction used. In general, it is more convenient to prepare recombinant proteins in soluble form. Purification steps vary with the production process and the particular protein produced. Often an oligopeptide can be produced from a chimeric nucleotide sequence. This is accomplished by ligating the vr4 nucleotide sequence or a desired portion thereof to a nucleotide sequence encoding a polypeptide domain which will facilitate protein purification (Kroll D J et al (1993) DNA Cell Biol 12:441-53).

In addition to recombinant production, fragments of VR4 may be produced by direct peptide synthesis using solid-phase techniques (eg Stewart et al (1969) Solid-Phase Peptide Synthesis, W H Freeman Co, San Francisco Calif.; Merrifield J (1963) J Am Chem Soc 85:2149-2154). Automated synthesis may be achieved, for example, using Applied Biosystems 431A Peptide Synthesizer (Foster City, Calif.) in accordance with the instructions provided by the manufacturer. Additionally, a particular portion of VR4 may be mutated during direct synthesis and combined with other parts of the peptide using chemical methods.

Antibodies specific for VR4 may be produced by inoculation of an appropriate animal with the polypeptide or an antigenic fragment. An antibody is specific for VR4 if it is produced against an epitope of the polypeptide and binds to a least part of the natural or recombinant protein. VR4 for antibody induction does not require biological activity; however, the protein must be antigenic. Peptides used to induce specific antibodies may comprise a portion of the VR4 sequence consisting of at least five aa, preferably at least 10 aa. An antigen portion of VR4 may be fused to another protein such as keyhole limpet hemocyanin, and the chimeric molecule used for antibody production.

Antibody production includes not only the stimulation of an immune response by injection into animals, but also analogous processes such as the production of synthetic antibodies, the screening of recombinant immunoglobulin libraries for specific-binding molecules (eg Orlandi R et al (1989) PNAS 86:3833-3837, or Huse W D et al (1989) Science 256:1275-1281) or the in vitro stimulation of lymphocyte populations. Current technology (Winter G and Milstein C (1991) Nature 349:293-299) provides for a number of highly specific binding reagents based on the principles of antibody formation. These techniques may be adapted to produce molecules which specifically bind VR4.

Various approaches may be utilised to raise monoclonal or polyclonal antibodies to VR4. In one approach, denatured protein from reverse phase HPLC separation is obtained in quantities up to 75 mg. This denatured protein is used to immunise mice or rabbits using standard protocols; about 100 μg are adequate for immunisation of a mouse, while up to 1 mg might be used to immunise a rabbit. For identifying mouse hybridomas, the denatured protein is radioiodinated and used to screen potential murine B-cell hybridomas for those which produce antibody. This procedure requires only small quantities of protein, such that 20 mg is sufficient for labelling and screening of several thousand clones.

In the second approach, the amino acid sequence of an appropriate VR4 domain, as deduced from translation of the cDNA, is analysed to determine regions of high antigenicity. Oligopeptides comprising appropriate hydrophilic regions are synthesised and used in suitable immunisation protocols to raise antibodies. Analysis to select appropriate epitopes is described by Ausubel F M et al (supra). The optimal amino acid sequences for immunisation are usually at the C-terminus, the N-terminus and those intervening, hydrophilic regions of the polypeptide which are likely to be exposed to the external environment when the protein is in its natural conformation.

Typically, selected peptides, about 15 residues in length, are synthesised using an Applied Biosystems Peptide Synthesiser Model 431A using fmoc-chemistry and coupled to keyhole limpet hemocyanin (KLH; Sigma, St Louis Mo.) or other suitable antigen by reaction with M-maleimidobenzoyl-N-hydroxysuccinimide ester (MBS; Ausubel F M et al, supra). If necessary, a cysteine is introduced at the C- or N-terminus of the peptide to permit coupling to the antigen. Animals such as rabbits may be immunised with the peptide-KLH complex in complete Freund's adjuvant. The resulting antisera are tested for antipeptide activity by binding the peptide to plastic, blocking with 1% bovine serum albumin, reacting with antisera, washing and reacting with labelled (radioactive or fluorescent), affinity purified, specific goat anti-rabbit IgG.

Hybridomas are prepared and screened using standard techniques. Hybridomas of interest are detected by screening with labelled VR4 to identify those fusions producing the monoclonal antibody with the desired specificity. In a typical protocol, wells of plates (FAST; Becton Dickinson, Palo Alto Calif.) are coated during incubation with affinity purified, specific rabbit anti-mouse (or suitable antispecies Ig) antibodies at 10 mg/ml. The coated wells are blocked with 1% BSA, washed and incubated with supernatants from hybridomas. After washing the wells are incubated with labelled VR4 at 1 mg/ml. Supernatants with specific antibodies bind more labelled VR4 than is detectable in the background. Then clones producing specific antibodies are expanded and subjected to two cycles of cloning at limiting dilution. Cloned hybridomas are grown in tissue culture by standard methods. Monoclonal antibodies with affinities of at least 10⁸ M⁻¹, preferably 10⁹ to 10¹⁰ or stronger, are typically made by standard procedure as described in Harlow and Lane (1988) Antibodies: A Laboratory Manual, Cold Spring Harbor Laboratory, Cold Spring Harbor, N.Y.; and in Goding (1986) Monoclonal Antibodies: Principles and Practice, Academic Press, New York City.

U.S. Pat. No. 6,172,197 B1 and references quoted therein describes alternative means, based on recombinant DNA techniques, for the screening and harvesting of monoclonal antibodies.

Particular VR4 antibodies are useful for investigating signal transduction and the diagnosis of infectious or hereditary conditions which are characterised by differences in the amount or distribution of VR4 or downstream products of an active signalling cascade.

Diagnostic tests for VR4 include methods utilising an antibody and a label to detect VR4 in human body fluids, membranes, cells, tissues or extracts of such. The polypeptides and antibodies of the present invention are used with or without modification. Frequently, the polypeptides and antibodies are labelled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, chromogenic agents, magnetic particles and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced as shown in U.S. Pat. No. 4,816,567.

A variety of protocols for measuring soluble or membrane-bound VR4, using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site monoclonal-based immunoassay utilising monoclonal antibodies reactive to two non-interfering epitopes on VR4 is preferred, but a competitive binding assay may be employed. These assays are described, among other places, in Maddox, Del. et al (1983, J Exp Med 158:1211f).

Native or recombinant VR4 may be purified by immunoaffinity chromatography using antibodies specific for VR4. In general, an immunoaffinity column is constructed by covalently coupling the anti-VR4 antibody to an activated chromatographic resin.

Polyclonal immunoglobulins are prepared from immune sera either by precipitation with ammonium sulfate or by purification on immobilized Protein A (Pharmacia LKB Biotechnology, Piscataway N.J.). Partially purified immunoglobulin is covalently attached to a chromatographic resin such as CnBr-activated Sepharose (Pharmacia LKB Biotechnology). The antibody is coupled to the resin, the resin is blocked, and the derivative resin is washed according to the manufacturer's instructions.

A soluble VR4 containing preparation is passed over the immunoaffinity column, and the column is washed under conditions that allow the preferential absorption of VR4 (eg. high ionic strength buffers in the presence of detergent). Then, the column is eluted under conditions that disrupt antibody/protein binding (eg. a buffer of pH 2-3 or a high concentration of a chaotrope such as urea or thiocyanate ion), and VR4 is collected.

The VR4 receptor protein of the invention, or binding fragments thereof, is suitable for use in assay procedures for the identification of compounds which modulate the receptor activity. Modulating receptor activity, as described herein, includes the inhibition or activation of the receptor and also includes directly or indirectly affecting the normal regulation of the receptor activity. Compounds which modulate the receptor activity include agonists, antagonists and compounds which directly or indirectly affect the normal regulation of the receptor activity.

The VR4 receptor protein or fragment thereof used in such assays may be obtained from either recombinant or natural sources. In general, an assay procedure to identify VR4 receptor modulators will involve the VR4 receptor protein of the invention and a test compound or a sample which contains a putative VR4 receptor modulator. The test compound or sample may be tested directly on, for example, purified receptor protein (native or recombinant), subcellular fractions such as membrane preparations of receptor-producing cells (native or recombinant) containing the receptor protein, or whole cells (native or recombinant) expressing the receptor protein. The test compound or sample may be added to the receptor protein in the presence or absence of known labelled or unlabelled receptor ligand. The modulating activity of the test compound or sample may be determined by, for example, analyzing the ability of the test compound or sample to bind to the receptor, activate receptor activity, inhibit receptor activity, enhance or inhibit the binding of other compounds to the receptor, modify receptor regulation, or modify an intracellular activity.

Modulators identified by such assays are expected to be useful in the control or alleviation of pain, especially heat-mediated pain, arthritis pain and neuropathic pain, inflammation, neurodegeneration such as that associated with Alzheimer's disease, Parkinson's disease or ischemia, endocrine disorders, cardiovascular disease, bladder or bowel dysfunction, mood disorders (e.g. depression), obesity and cancer.

Thus, the present invention provides methods of screening for drugs or any other agents which affect VR4 signal transduction. In one such method, the VR4 receptor protein or fragment thereof is first contacted with a ligand of known affinity for the VR4 receptor, and then with the test compound, and the ability of the test compound to compete with the known ligand in binding to the receptor is measured. Typically, the known ligand is labelled (e.g. with a radioactive isotope or a fluorescent moiety) to facilitate its detection and quantitation.

Advantageously, parallel screening of the same test compounds for affinity to the VR1 and/or other related Trp receptors may be carried out, enabling the identification of compounds having a selective affinity for the VR4 receptor. Parallel screening may also be used to identify compounds combining a low affinity to VR4 with a high affinity to VR1 and/or other Trp receptors.

High throughput screening of test compounds may be achieved using Aurora reporter assays such as fluorescent imaging with Ca-sensitive dyes (such as Fluo3, Fluo4 or Fura2) on equipment such as the FLIPR (fluorometric imaging plate reader) or the VIPR (voltage ion probereader).

Another technique for drug screening, adaptable for high throughput screening for compounds having binding affinity to the VR4 polypeptides, is described in detail in International Patent Publication WO84/03564, published on Sep. 13, 1984. Briefly stated, large numbers of different small peptide test compounds are synthesised on a solid substrate, such as plastic pins or some other surface. The peptide test compounds are reacted with VR4 polypeptide and washed. Bound VR4 polypeptide is then detected by methods well known in the art.

Purified VR4 may also be coated directly onto plates for use in the aforementioned drug screening techniques. In addition, non-neutralising antibodies may be used to capture the peptide and immobilise it on a solid support.

This invention also contemplates the use of competitive drug screening assays in which neutralising antibodies capable of binding VR4 specifically compete with a test compound for binding to VR4 polypeptides or fragments thereof. In this manner, the antibodies are used to detect the presence of any peptide which shares one or more antigenic determinants with VR4.

The goal of rational drug design is to produce structural analogues of biologically active polypeptides of interest or of small molecules with which they interact, agonists, antagonists, or inhibitors. Any of these examples are used to fashion drugs which are more active or stable forms of the polypeptide or which enhance or interfere with the function of a polypeptide in vivo (eg. Hodgson J (1991) Bio/Technology 9:19-21).

In one approach, the three-dimensional structure of a protein of interest, or of a protein-inhibitor complex, is determined by x-ray crystallography, by computer modelling or, most typically, by a combination of the two approaches. Both the shape and charges of the polypeptide must be ascertained to elucidate the structure and to determine active site(s) of the molecule. Less often, useful information regarding the structure of a polypeptide is gained by modelling based on the structure of homologous proteins. In both cases, relevant structural information is used to design efficient inhibitors. Useful examples of rational drug design includes molecules which have improved activity or stability as shown by Braxton S and Wells J A (1992, Biochemistry 31:7796-7801) or which act as inhibitors, agonists, or antagonists of native peptides as shown by Athauda S B et al (1993 J Biochem 113:742-46), incorporated herein by reference.

It is possible to isolate a target-specific antibody, selected by functional assay, as described above, and then to solve its crystal structure. This approach, in principle, yields a pharmacore upon which subsequent drug design is based. It is possible to bypass protein crystallography altogether by generating anti-idiotypic antibodies (anti-ids) to a functional, pharmacologically active antibody. As a mirror image of a mirror image, the binding site of the anti-ids is expected to be an analogue of the original receptor. The anti-id is then used to identify and isolate peptides from banks of chemically or biologically produced peptides. The isolated peptides then act as the pharmacore.

By virtue of the present invention, sufficient amount of polypeptide are made available to perform such analytical studies as X-ray crystallography. In addition, knowledge of the VR4 amino acid sequence provided herein provides guidance to those employing computer modelling techniques in place of or in addition to x-ray crystallography.

The inventive purified VR4 is a research tool for identification, characterisation and purification of interacting G-protein or other signal transduction pathway proteins. Radioactive labels are incorporated into a selected VR4 domain by various methods known in the art and used in vitro to capture interacting molecules. A preferred method involves labelling the primary amino groups in VR4 with ¹²⁵I Bolton-Hunter reagent (Bolton, A E and Hunter, W M (1973) Biochem J 133: 529). This reagent has been used to label various molecules without concomitant loss of biological activity (Hebert C A et al (1991) J Biol Chem 266: 18989; McColl S et al (1993) J Immunol 150:4550-4555).

Labelled VR4 is useful as a reagent for the purification of molecules with which it interacts. In one embodiment of affinity purification, membrane-bound VR4 is covalently coupled to a chromatography column. Cell-free extract derived from synovial cells or putative target cells is passed over the column, and molecules with appropriate affinity bind to VR4. VR4-complex is recovered from the column, and the VR4-binding ligand disassociated and analysed, e.g. by N-terminal protein sequencing, proteomics/mass spectrometry or HPLC/mass spectrometry. Amino acid sequences thus identified can be used to design degenerate oligonucleotide probes for cloning the relevant genes from appropriate cDNA libraries.

In an alternative method, antibodies are raised against VR4, specifically monoclonal antibodies. The monoclonal antibodies are screened to identify those which inhibit the binding of labelled VR4. These monoclonal antibodies are then used therapeutically.

Bioactive compositions comprising agonists, antagonists, or antibodies of VR4 may be administered to human or animal subjects in a suitable therapeutic dose determined by any of several methodologies including clinical studies on mammalian species to determine maximal tolerable dose and on normal human subjects to determine safe dose. Additionally, the bioactive agent may be complexed with a variety of well established compounds or compositions which enhance stability or pharmacological properties such as half-life

Antibodies, inhibitors, or antagonists of VR4 (or other treatments to limit signal transduction, LST) provide different effects when administered therapeutically. LSTs are formulated in a nontoxic, inert, pharmaceutically acceptable carrier medium. An aqueous carrier medium is preferably at a pH of about 5 to 8, more preferably 6 to 8, although pH may vary according to the characteristics of the antibody, inhibitor, or antagonist being formulated and the condition to be treated. Characteristics of LSTs include solubility of the molecule, half-life and antigenicity/immunogenicity. These and other characteristics aid in defining an effective carrier. Native human proteins are preferred as LSTs, but organic or synthetic molecules resulting from drug screens are equally effective in particular situations.

LSTs are delivered by known routes of administration including but not limited to topical creams and gels; transmucosal sprays and aerosols; transdermal patches and bandages; injectable, intravenous and lavage formulations; and orally administered liquids and pills particularly formulated to resist stomach acid and enzymes. The particular formulation, exact dosage, and route of administration is determined by the attending physician and varies according to each specific situation.

Such determinations are made by considering multiple variables such as the condition to be treated, the LST to be administered, and the pharmacokinetic profile of a particular LST. Additional factors which are taken into account include severity of the disease state, patient's age, weight, gender and diet, time and frequency of LST administration, possible combination with other drugs, reaction sensitivities, and tolerance/response to therapy. Long acting LST formulations might be administered every 3 to 4 days, every week, or once every two weeks depending on half-life and clearance rate of the particular LST.

Normal dosage amounts vary from 0.1 to 100,000 μg, up to a total dose of about 1 g, depending upon the route of administration. Guidance as to particular dosages and methods of delivery is provided in the literature; see U.S. Pat. Nos. 4,657,760; 5,206,344; or 5,225,212. Those skilled in the art employ different formulations for different LSTs. Administration to cells such as nerve cells necessitates delivery in a manner different from that to other cells such as vascular endothelial cells.

It is contemplated that abnormal signal transduction, trauma, or diseases which trigger VR4 activity are treatable with LSTs. These conditions or diseases are specifically diagnosed by the tests discussed above, and such testing should be performed in suspected cases of viral, bacterial or fungal infections; allergic responses; mechanical injury associated with trauma; hereditary diseases; lymphoma or carcinoma; or other conditions which activate the genes of lymphoid tissues.

An additional embodiment of the subject invention is the use of VR4 specific antibodies, inhibitors or antagonists as bioactive agents to treat pain, especially heat-mediated pain, arthritis pain and neuropathic pain, inflammation, neurodegeneration such as that associated with Alzheimer's disease, Parkinson's disease or ischemia, endocrine disorders, cardiovascular disease, bladder or bowel dysfunction, mood disorders (e.g. depression), obesity and cancer.

The examples below are provided to illustrate the subject invention.

### EXAMPLES

### Example 1

Identification and Cloning of the Gene Encoding Human VR4 Receptor

Homology searches using the BLAST2 algorithm (Altschul S F et al, Nucleic Acids Res. (1997) 25, 3389-402) and the human protein sequence of the VR1 receptor were performed on the human HTG (High Throughput Genomic) phase 0-2 sequences of Genbank, contained in the Merck.HTG.Human database. A number of partial genomic contigs contained sequences homologous to, but not identical to any other known vanilloid, or Trp receptors. The regions of homology were identified on at least the following GenBank accession numbers (as at Oct. 20, 2000); ac027040, ac025125, ac027796, ac040891. These genomic sequences correspond to the chromosomal location 17p13.2 on human chromosome 17, adjacent to the chromosomal location of the human VR1 gene. Putative exons of VR4 as represented by regions of homology to the human VR1 protein sequence were assembled in the order that they would occur in VR1 to give a predicted protein sequence of 621 amino acids, but that lacked a start methionine and amino-terminal sequence, two internal exons, and a carboxy-terminal sequence.

The sequence was extended at the 5′ and 3′ ends by 5′ and 3′ RACE and anchored PCR using primers based on multiple sequences predicted using the Genehunt algorithm (Compugen). Exon prediction with Genehunt utilised the NCBI genomic contig NT_{—}010816 (GI: 13653147). All PCR amplifications used first strand cDNA from human brain or spinal cord (Clontech), or from RNA prepared from the human neuroblastoma SK-N-DZ (ATCC #CRL-2149). The sequence of the missing internal exons not predicted by the initial homology searches were also obtained by RT-PCR from the same RNA sources. The nucleotide sequences of the oligonucleotide primers used in for the PCR reactions are listed below:

| | | SEQ |
|---|---|---|
| Primer | Sequence (5′-3′) | ID NO |
| oligo1 | GAAGACGCACGTCTCCTTCCTTAAC | 3 |
| oligo2 | TTCATAGGCCTCCTCTGTGTACTCG | 4 |
| oligo3 | GCGGTAGTACGAGACGAGGGTCA | 5 |
| oligo4 | CATGAGATGCTGACCCTGGAGCC | 6 |
| oligo5 | AGTACCTGTTCGTCTTACAGGCCCC | 7 |
| oligo6 | GCGTGGAGGAGTTGGTAGAGTTGC | 8 |
| oligo7 | AGGCACATCCTCATCATGGCG | 9 |
| oligo8 | ATGAAAGCCCACCCCAAGGAGATG | 10 |
| oligo9 | ACCAGAGATGCACTGCCGGATGTT | 11 |
| oligo10 | CGCTTCTTCAGCCGCCTCTTTTTC | 12 |
| oligo11 | TCCATGGGCTTGGAGAAGACAGGA | 13 |
| oligo12 | AGATGCACTGCCGGATGTTGGAAT | 14 |
| oligo13 | CCATCCATCTCTCCCAGCCAAGCCGAC | 15 |
| oligo14 | CCTCCTCCCCCAGGATATCCAGCTTAC | 16 |
| oligo15 | AATTGGTAAAACCAGAGGCTTCACCCG | 17 |
| oligo16 | TTGCTCTGTGGAGGTCAAAACTCTTGGA | 18 |
| oligo17 | TGCTGGGCTCTCCTAGGACCATAGCATT | 19 |
| oligo18 | GAAGAAGGATTGGTGAACTGGGAAGGGA | 20 |

DNA consisting of some 5′ untranslated sequence, the putative coding region and some 3′ untranslated region was amplified in four segments by RT-PCR from whole brain and neuroblastoma first strand cDNA, and assembled sequentially into the cloning vector pCR-II-TOPO (Invitrogen) as four fragments, cut appropriately with the restriction enzymes NdeI, XhoI, BamHI, BspEI and HindIII. Once assembled, and verified by DNA sequencing, the construct was recloned into the pIRES-eGFP vector (Clontech) as a ApaI-SacI fragment. The sequences of the oligonucleotide primers are listed below:

| | | SEQ |
|---|---|---|
| Primer | Sequence (5′-3′) | ID NO |
| oligo19 | GTTGATGAACCTGCCCAGGATGT | 21 |
| oligo20 | TCAACCCCAACACCAAGGAGATAG | 22 |
| oligo3 | GCGGTAGTACGAGACGAGGGTCA | 5 |
| oligo4 | CATGAGATGCTGACCCTGGAGCC | 6 |
| oligo5 | AGTACCTGTTCGTCTTACAGGCCCC | 7 |
| oligo14 | CCTCCTCCCCCAGGATATCCAGCTTAC | 16 |
| oligo17 | TGCTGGGCTCTCCTAGGACCATAGCATT | 19 |

The human VR4 receptor protein is predicted to be 790 amino acids in length.

The preferred open reading frame and predicted amino acid sequence are shown in SEQ ID NO: 1 (FIG. 1) and SEQ ID NO: 2 (FIG. 2) respectively. However, analysis of the DNA sequence 5′ of the preferred start ATG codon reveals an additional in-frame ATG codon. Analysis of the nucleotide frequencies at this potential ATG (Kozak sequence analysis) suggests that it is not the likely initiation codon. However, usage of this codon would add the additional sequence MGPLNSLKLPLSPRPEHLPCGCIPA (SEQ ID NO: 23) to the amino terminal of the VR4 sequence presented in SEQ ID NO: 2. Additionally, the sequence shown in SEQ ID NO: 1 may be subject to both naturally occurring and artificially introduced variations, polymorphisms and mutations, including, but not limited to one or more of the following: G to A change at position 154, A to G change at position 351, A to C change at position 639, or a T to C change at position 2099. These changes may result in a change to the amino acid sequence of the VR4 receptor protein shown in SEQ ID NO: 2, as in the G to A change at base 154 (change of amino acid from Valine [V] to Isoleucine [I]), and the T to C change at base 2099 (change of amino acid from Leucine [L] to Proline [P]), or the change may be silent (i.e. the amino acid sequence is unchanged).

Messenger RNA expression of the putative gene was confirmed by RT-PCR from adult brain (Clontech) using the oligonucleotide primers 5′-GGGCCTTCTTCAACCCCAAG (SEQ ID NO: 24) and 5′-AACTTCCTGGACAGGCTCCG (SEQ ID NO: 25). A band of the predicted size of 391 bp was cloned and confirmed by DNA sequencing and by Southern blotting (FIG. 3(a)). Distribution of the messenger RNA was also examined by RT-PCR in a human adult tissues array (Clontech) (FIG. 3(b)). In addition, the expression pattern was evaluated using a Multiple Tissue DotBlot (Clontech) (FIG. 3(c)), using the oligonucleotide probes listed below.

The results indicate that mRNA encoding VR4 is expressed in human dorsal root ganglia and central nervous system, including cortex, corpus callosum and thalamus.

| Probe | DNA Sequence (5′-3′) | SEQ ID NO |
|---|---|---|
| 1 | GCAAAGGCAAGCAGGATCCGCACTATCT | 26 |
| | CCTTGGTGTTGGGGTGG | |
| 2 | ATGTCCGTCTGCTCGTGCTCCATCAGCA | 27 |
| | GCTGCACAATCTCGGGC | |
| 3 | GCTTGAAGAGTTCCAGCACTGCGTCGCT | 28 |
| | GAAGCTGCCGTAGGAGC | |

Analysis of the VR4 sequence suggests that it is a member of the oTrp family of Trp receptors, and shares 43% sequence identity with the vanilloid receptor VR1, and 42% sequence identity with the human oTrpc4 receptor. In comparison, VR1 and oTrpc4 share 46% sequence identity. A multiple sequence alignment is shown in FIG. 4 (in which VR2 represents the VRL-1 receptor, and VR3 represents the oTrpC4 receptor) and a dendrogram illustrating the sequence similarities of the extended Trp family is shown in FIG. 5. The dendrogram was generated using a clustalw multiple sequence alignment produced with the settings:

pair-wise gap creation=13, pairwise gap extension=0.2, pairwise matrix=identity; multiple gap creation=15, multiple gap extension=0.02, multiple matrix=identity.

### Example 2

Mouse VR4

The DNA and predicted amino acid sequence for the murine VR4 gene was assembled by homology searches (blast2:tblastn) of the mouse genomic sequence databases (Merck.GENOMIC.MSC.00, Merck.GENOMIC.MSC.01, Merck.GENOMIC.MSC.02, Merck.GENOMIC.MSC.03, Merck.GENOMIC.MSC.04) using the protein sequences corresponding to the individual exons of human VR4 as a query sequence. DNA and predicted amino acid sequence of murine VR4 are shown in SEQ ID NO: 29 (FIG. 6) and SEQ ID NO: 30 (FIG. 7) respectively.

A DNA sequence alignment between the genes for human VR4 (top line) and mouse VR4 (bottom line) is shown in FIG. 8.

An amino acid sequence alignment between human VR4 (top line) and mouse VR4 (bottom line) is shown in FIG. 9.

### Example 3

Antibodies

The sequence RTDFNKIQDSSRNNSKT from human VR4 was selected as an antigenic peptide for polyclonal antibody production in rabbits. A synthetic peptide with the sequence RTDFNKIQDSSRNNSKTC (SEQ ID NO: 31) was synthesised and conjugated to KLH for immunisation using standard techniques.

### Example 4

Functionality

Elucidation of VR4 function was undertaken by electrophysiological recording. A glass coverslip containing a monolayer of (HEK293 or CHO) cells transiently transfected with the human VR4 receptor in the pIRES-eGFP vector was placed in a perspex chamber mounted on the stage of an inverted phase-contrast microscope and continuously perfused with Modified A solution (see below).

Fire-polished patch pipettes were pulled using conventional 120TF-10 electrode-glass. Pipette tip diameter was generally 1.0-2.0 μm, and resistances were approximately 2-3 MΩ. The intracellular pipette solution used is detailed below.

A 20 msec, 1 mV voltage command was applied to the pipette at a frequency of 2 Hz and the current amplitude continuously monitored using pClamp hardware and software (8.0) and an oscilloscope. Positive internal pressure was applied to the pipette (prior to lowering into the bath) which was then advanced upon the cell under study using a Burleigh patch-clamp driver. After touching the pipette tip against the cell membrane, the positive pressure was released from the pipette and gentle suction applied. At this stage the voltage pulse did not elicit a noticeable pipette current indicating an increase in tip resistance.

A continuous voltage command was then applied to the pipette in order to voltage clamp the membrane patch to −60 mV and the voltage pulse was increased to 10 mV. This typically led to the formation of a seal resistance in excess of 1 GΩ. The membrane patch within the tip of the pipette was then ruptured by the further application of suction as was apparent from a dramatic increase in capacitive transients due to the increased membrane time constant, and a fall in apparent pipette resistance. Capacitive transients and series resistance were compensated.

Solutions with different drugs or different compositions were applied to the cell under study for 5 to 60 seconds, followed by a 30-120 second wash period by fast perfusion using a large internal diameter (500 μm) triple-barrel pipette assembly. The top barrel contained the wash solution, the other two barrels contained the various test solutions. Responses to solution changes were obtained by rapidly switching the position of the perfusion pipette in order to envelop the recorded cell completely in drug solution. This was achieved via a Biologic rapid solution changer to pivot the barrels into the desired position. Fast washout was obtained by re-positioning the washout barrel in line with the cell.

A voltage step (from −60 mV to −80 mV for 50 ms followed by a voltage ramp to +80 mV within 500 ms and a 50 ms step (at +80 mV) before returning to −60 mV, or a ramp from −60 to +80 mV for 500 ms then returning to −60 mV, was applied 3s before and during application of the test solution. This protocol increased the sensitivity of the assay.

Functional activation of VR4 transfected cells was obtained by application of heated modified A solution warmed using a ramp protocol from room temperature to 45° C. An example of the recordings from a CHO cell transiently transfected with human VR4 in the pIRES-eGFP vector (A1), and a cell transiently transfected with empty pIRES-eGFP vector (B) is shown in FIG. 10. The application of heated modified A solution (45° C.) is indicated by the horizontal bar above the trace. Also indicated on the example trace recordings is the current induced by a voltage ramp from −60 to +80 mV (vertical lines). The dashed line indicates zero current. The inset (A2) shows the current-voltage relationship of the heat-induced current shown in A1, with some outward rectification.

A summary of the heat induced and leak currents of cells transfected with human VR4 and mock (empty vector) transfected cells is shown below.

| | Heat Induced Peak Current [pA] | | Leak Current [pA] | |
|---|---|---|---|---|
| | VR4 | Mock | VR4 | Mock |
| Mean | −158.9 | −24.2 | −176.7 | −54.7 |
| SEM | 40.2 | 3.9 | 50.0 | 19.9 |
| n | 10 | 8 | 10 | 8 |
| p= | 0.0085 | | 0.0433 | |

| | mM | MW | g/l | g/21 |
|---|---|---|---|---|
| CaCl₂.2H₂O | 1.67 | 147.02 | 0.246 | 0.491 |
| MgCl₂.6H₂O | 1 | 203.3 | 0.203 | 0.407 |
| KCl | 2 | 74.55 | 0.149 | 0.298 |
| NaCl | 165 | 58.44 | 9.643 | 19.285 |
| D-glucose | 17 | 180.16 | 3.063 | 6.125 |
| HEPES | 10 | 238.3 | 2.383 | 4.766 |

pH (NaOH) 7.3

Composition of Cesium Fluoride Intracellular Pipette Solution:

| | MW | mM | g/100 ml |
|---|---|---|---|
| CsF | 172.4 | 110 | 1.894 |
| TEAC1 | 165.7 | 30 | 0.497 |
| Cs-BAPTA | 1004.3 | 20 | 2.008 |
| ATP-Mg | 507.2 | 2 | 0.101 |
| MgCl₂.6H₂O | 1 M | 1 | 100 μl |
| HEPES | 238.3 | 10 | 0.2383 |

pH (TEA-OH) 7.2
˜315 mOsm

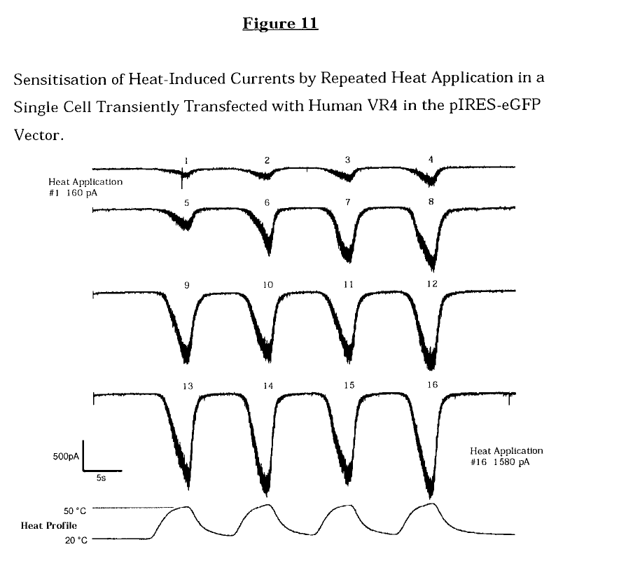

The effect of the repeated application of heated modified A solution heated using a ramp protocol from room temperature to 50° C. is shown in FIG. 11. In this example, a single CHO cell transiently transfected with human VR4 in the pIRES-eGFP vector was subjected to 4 groups of 4 heat ramps from ambient (20° C.) to 50° C. and back to ambient as depicted in the Heat Profile trace. The recordings of these 16 heat applications is shown. The current amplitudes increase from 160 pA for the first application to 1580 pA in heat application number 16.

1. A purified polynucleotide comprising a nucleic acid sequence encoding the polypeptide of SEQ ID NO: 2, or the complement of said polynucleotide.

2. The polynucleotide of claim 1 comprising the nucleic acid sequence of SEQ ID NO: 1.

3. An antisense molecule comprising the complement of the polynucleotide of claim 2 or a portion thereof.

4. A pharmaceutical composition comprising the antisense molecule of claim 3 and a pharmaceutically acceptable excipient.

5. A diagnostic composition comprising an oligomer of the polynucleotide of claim 2.

6. A diagnostic test for a condition associated with altered VR4 expression comprising the steps of:
a) providing a biological sample;
b) combining the biological sample and the diagnostic composition of claim 5;
c) allowing hybridisation to occur between the biological sample and the diagnostic composition under suitable conditions;
d) measuring the amount of hybridisation to obtain a sample value; and
e) comparing the sample value with standard values to determine whether vr4 expression is altered.

7. An expression vector comprising the polynucleotide of claim 1.

8. A host cell transformed with the expression vector of claim 7.

9. A method of producing a polypeptide, said method comprising the steps of:
a) culturing the host cell of claim 8 under conditions suitable for the expression of the polypeptide; and
b) recovering the polypeptide from the host cell culture.

10. A purified polypeptide (VR4) comprising the amino acid sequence of SEQ ID NO: 2.

11. A diagnostic composition comprising the polypeptide of claim 10 or a portion thereof.

12. A pharmaceutical composition comprising the polypeptide of claim 10 and a pharmaceutically acceptable excipient.

13. An antibody specific for the purified polypeptide of claim 10, or for a portion of that polypeptide.

14. A diagnostic composition comprising the antibody of claim 13.

15. A diagnostic test for a condition associated with altered VR4 expression comprising the steps of:
a) providing a biological sample;
b) combining the biological sample and the antibody of claim 13 under conditions suitable for complex formation;
c) measuring the amount of complex formation between VR4 and the antibody to obtain a sample amount; and
d) comparing the amount of complex formation in the sample with standard amounts of complex formation, wherein a variation between sample amount and standard amounts of complex formation establishes the presence of the condition.

16. A method for screening one or more test compounds as modulators of VR4 receptor signal transduction comprising the steps of:
(a) contacting each test compound with a polypeptide comprising the amino acid sequence of SEQ ID NO: 2; and
(b) measuring the modulatory effect of each test compound.

17. The method of claim 16 wherein said polypeptide is contained in natural or recombinant whole cells expressing the polypeptide, or in subcellular fractions derived therefrom.

18. A method according to claim 16 or claim 17 wherein the modulatory effect comprises binding of the test compound to the polypeptide.

19. A method according to claim 18 wherein the test compounds either have a high affinity for at least one Trp receptor other than VR4, or have a low affinity for at least one Trp receptor other than VR4.

20. A pharmaceutical composition comprising a pharmaceutically acceptable excipient and a compound which is a modulator of VR4 receptor signal transduction, which compound being identified by the method of claim 16.

21. A pharmaceutical composition comprising a pharmaceutically acceptable excipient and a compound which either:
(a) binds selectively to the VR4 receptor in preference to at least one other Trp receptor; or
(b) binds selectively to at least one other Trp receptor in preference to VR4;
which compound being identified by the method of claim 19.
